(19) United States
(12) Patent Application Publication
(10) Pub. No.: US 2016/0117957 A1
(43) Pub. Date: Apr. 28, 2016

(54) Peel Back Fragrance Label for Scent Sampling

(71) Applicants: John DiGregorio, Brick, NJ (US); Shawn Borman, Pennsauken, NJ (US)

(72) Inventors: John DiGregorio, Brick, NJ (US); Shawn Borman, Pennsauken, NJ (US)

(21) Appl. No.: 14/121,835

(22) Filed: Oct. 23, 2014

(51) Int. Cl. G09F 3/02; B65C 9/38; B65C 9/18; A61L 9/12; A45D 40/00

(52) U.S. Cl. CPC G09F 3/02; A61L 9/12; A45D 40/0087; B65C 9/18; B65C 9/38; G09F 2003/028; G09F 2003/0257

(57) ABSTRACT

A scent/fragrance delivery system in the form of a peel back label in which a fragrance/oil of a scented nature is encapsulated in a carrier, such as a plastic cell wall, the plastic encapsulation protecting microscopic beads or oil containing the scent/fragrance and in cooperation with opposing outer substrate layers, deters the scent/fragrance from escaping or migrating until its intended use, the scent/fragrance activated by separating the two opposing substrates by the peel back method which ruptures or breaks open the encapsulation of the beads of oil containing the scent/fragrance, the delivery system capable of use as a single use system or a reusable system.

### RELATED APPLICATIONS

Applicant claims the benefit of provisional application Ser. No. 61/901,019, filed Nov. 7, 2013.

### BACKGROUND OF THE INVENTION

1. Field of the Invention

The present invention relates to a scent/fragrance sampling label, in which a fragrance or scented oil is encapsulated in a carrier and applied between two outer substrate layers, the separation of the outer substrate layers causing the release of fragrance or scent from its microencapsulated fragrance oil or scent infused coated substrate.

2. Description of the Prior Art

Manufacturers of consumer products have a profound interest in determining the acceptability of their products by the consuming public. For that reason, manufacturers routinely test market new products with consumers in order to get their reactions. The test marketing can involve a myriad of trials. The manufacturers can hire representatives to offer samples of the new products at the locations where the new products might be sold. These samples would be provided gratis to customers and then the customers would be asked their reaction. Additionally, the manufacturer may have a mass mailing of small samples of the new product to consumers in a particular geographic location, together with a questionnaire regarding the consumer's likes or dislikes with respect to the product.

The products involved in such consumer testing can range from fine fragrances and colognes, health care products, such as hair shampoos and conditioners, to makeup, to kitchen cleaners, all purpose cleaners, dishwashing cleaners, clothes and soap blends, and health care, as well as beauty care products. The manufacturers are interested not only in how the consumer reacts to the new product and its effectiveness, but also if the consumer is influenced by the style and even the scent or fragrance of the product. Thus health care and beauty care products are test marketed extensively. An individual's most common interaction with a test marketing situation involves a scent or fragrance and occurs at the perfume or mens cologne counter of large department stores. There are numerous sample bottles of cologne, perfume, and eau de toilette on display for sampling. In some instances, these are actual bottles of the product with small absorbent substrates associated therewith where one can spray the scent of a particular product onto the absorbent substrate, and wave the substrate and then smell the substrate to obtain a sense of the fragrance or scent of the particular perfume. In other instances, the manufacturers go to great expense to have extremely small samples of the product prepared which they can give away as promotional materials to people in order to gain their feedback regarding the fragrance and scent.

The aforesaid market testing can be extremely expensive to the manufacturer, both from a manufacturing context in having to manufacture small sampling examples of the product, mailing costs for such type of samples, and man power costs. Therefore there has been a need for a scent/fragrance delivery system which is inexpensive to manufacture and can be mailed inexpensively or distributed very inexpensively which will allow the consumer to sample the product from a scent/fragrance standpoint.

### OBJECTS OF THE INVENTION

An object of the present invention is to provide for a novel scent/fragrance delivery system for inexpensively providing sample scents and fragrances to the consumer for sampling purposes.

A still further object of the present invention is to provide for a novel scent/fragrance delivery system in the form of peel back fragrance labels which can be manufactured inexpensively in a variety of shapes and sizes.

A further object of the present invention is to provide for a novel scent/fragrance delivery system which does not require an additional adhesive in order to seal the peel back label closed.

A still further object of the present invention is to provide for a novel scent/fragrance delivery system which when opened, leaves some of the encapsulated fragrance/scent unbroken and secured to one of the substrates such that it can be later scratched or rubbed for additional scent/fragrance sampling.

A still further object of the present invention is to provide for a novel scent/fragrance delivery system wherein the substrates are printable and suitable substrates can be chosen from a variety of materials of varying thickness to achieve the look and feel desired by the manufacturer.

A still further object of the present invention is to provide for a novel scent/fragrance delivery system in which the encapsulated scent/fragrance bonds with the inner sides of the top and bottom substrates holding the two-ply together without an adhesive layer until removal or separation of the two substrates is desired.

A still further object of the present invention is to provide for a novel scent/fragrance delivery system which has a long shelf life.

A still further object of the present invention is to provide for a novel scent/fragrance delivery system which is economical to fabricate and which eliminates the adhesive station in the fabrication process, and is adaptable to both narrow web and wide web manufacture.

A still further object of the present invention is to provide for a novel scent/fragrance delivery system requiring no additional adhesive and thereby allowing the scent/fragrance greater surface area during the application stage.

A still further object of the present invention is to provide for a novel scent/fragrance delivery system which not only allows the consumer to separate the substrates to sample the scent/fragrance from the product, but also allows the consumer to wipe or rub the exposed substrate and scent/fragrance onto the user's skin for sampling.

A still further object of the present invention is to provide for a novel scent/fragrance delivery system which provides the product owner, with a greater flexibility in the manner in which he can present the sample to the consuming public.

### SUMMARY OF THE INVENTION

A scent/fragrance delivery system in the form of a peel back label in which a fragrance/oil of a scented nature is encapsulated in a carrier, such as a plastic cell wall, the plastic encapsulation protecting microscopic beads or oil containing the scent/fragrance and in cooperation with opposing outer substrate layers, deters the scent/fragrance from escaping or migrating until its intended use, the scent/fragrance activated by separating the two opposing substrates by the peel back method which ruptures or breaks open the encapsulation of the beads of oil containing the scent/fragrance, the delivery system capable of use as a single use system or a reusable system.

### BRIEF DESCRIPTION OF THE DRAWINGS

These and other objects of the present invention will become apparent, particularly when taken in light of the following illustrations wherein:

FIG. 1 is a side view of the peel back label illustrating the various layers of its composition;

FIG. 2 is a top view of the peel back fragrance label;

FIG. 3 is a schematic of the assembly line for the peel back fragrance label;

FIG. 4 is a top view of the peel back fragrance label as fabricated on a narrow web flexographic print press one label/lane across; and

FIG. 5 is a top view of the peel back fragrance label as fabricated on a narrow web flexographic printing press producing multiple units/lanes across.

### DETAILED DESCRIPTION OF THE INVENTION

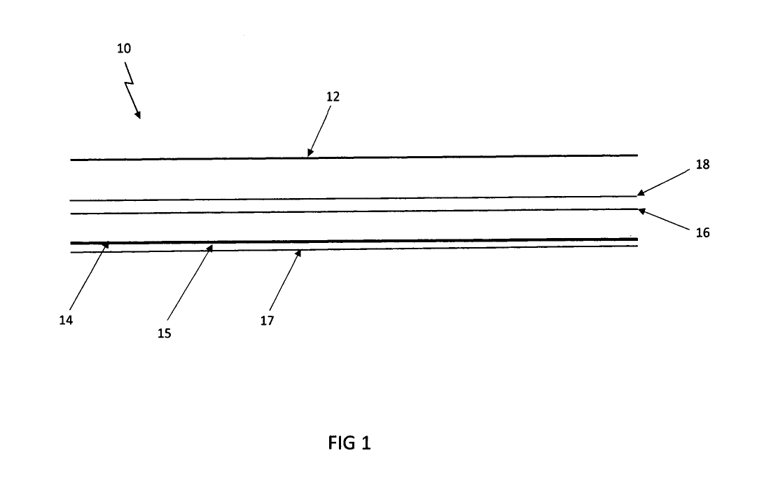

FIG. 1 illustrates the side view of the various layers used to compose the peel back fragrance label 10. There are two exterior substrates forming the label. Upper substrate 12 and lower substrate 14 may be composed of the same type of material or preference may dictate that one substrate be of a different material than the other. For its intended purpose, it has been found that a transparent polypropylene film of suitable thickness is suited for upper substrate 12 and a like substrate of equal or greater thickness is suited for the lower substrate 14. Lower substrate could also be of a paper material or other suitable cellulose material, as well as a polymer of greater density and thickness than upper substrate 12, some of which may require a primer to be applied and dried prior to the fragrance application. The upper substrate being transparent or opaque allows for graphics or text whichever is desired to be printed directly on either surface or side of the substrate. Similarly, such graphics or text printing could also be applied to the lower substrate.

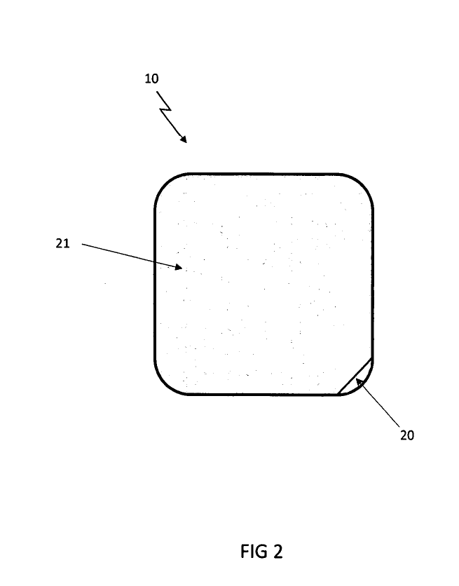

To aid in the manufacturing process and the fabrication of the peel back fragrance label, the lower substrate 16 is also associated with an adhesive layer 15 on its lower outer surface and a liner 17 which sandwiches the adhesive layer 15 between the lower substrate and the liner which is removable. This structure of the lower substrate 16, outer adhesive layer 15, and removable liner 17 is the manner in which the lower substrate, adhesive layer, and liner are removed from a roll of material and form a web as it proceeds through the printing and coating process as explained at length and in detail hereafter. A portion of the dual substrate is partially removed at the conclusion of the coating and drying process leaving separate peel back fragrance labels affixed to the release liner 17. The individual peel back fragrance label can then be removed from the liner, exposing the adhesive layer which can be used to attach the peel back fragrance label to a sample of the product or similar structure so that a user can then peel back the upper substrate 12 to release the fragrance as more fully explained with respect to FIG. 2.

The fragrant composition applied to or coated on the lower substrate 14 consists of two types of slurry. The first slurry applied forming the first layer 16 is a water based fragrance slurry. The second slurry which is applied is a UV based fragrance slurry 18. The water based fragrance slurry 16 is first applied to the surface of the lower substrate 14 and then immediately subjected to hot air drying. The next layer applied, the UV based fragrance slurry 18 is applied over the top of the now dried water based fragrance slurry 16, and then the upper substrate layer 12 is applied over the UV fragrance slurry and secured and conjoined with the lower substrate 16. The conjoined peel back label is then exposed to a UV heat lamp which cures both fragrance layers 16 and 18 and melds them together, and creates a bond with top substrate 12 with the UV slurry.

The fragrance oil utilized with respect to the water based fragrance slurry 16 and the UV based fragrant slurry 18 may be selected from a wide variety of stock fragrance oils utilized in the perfume, cologne, and cleaning industry. The oils may be singular or a mixture of several components to achieve the desired fragrance. They contain a minimum of volatile or water soluble reactive ingredients to help the fragrance oils become encapsulated in their respective slurries forming a cell wall around the fragrance oil creating beads of microencapsulated fragrance. The slurry is a mix of encapsulated fragrance oil/beads and a transporter fluid to enable the transfer of the fragrance beads to the host substrate, lower substrate 14. The curing of the slurries by UV exposure flashes off the transporter solution leaving a substantially dried fragrance encapsulated layer. The microencapsulated fragrance beads will bind with the inner surfaces of the bottom substrate 14 and top substrate 12 and remain intact until the upper substrate is peeled back from the peel back label.

FIG. 2 is a top view of a single peel back fragrance label 10. It is evident from the top view that the absence of the need for any adhesive in order to secure the upper substrate 12 and lower substrate 14 by the fragrance slurries alone allows for the surface of the lower substrate 14 to be substantially completely coated with the slurry compositions 16 and 18 leaving only a corner 20 purposely left uncoated. This allows for a corner tab of the upper substrate 12 to be free from the coated slurries and the lower substrate 14. It is this corner tab 20 that is utilized by the user to peel back the upper substrate 12 to simultaneously rupture some microencapsulated fragrance beads and to release the fragrance or scent for testing. The lift tab can be positioned in any of the four corners. The microencapsulated fragrance beads are referenced and depicted by dots 21 for purposes of explanation, it being understood that in actuality they would not be visible to the naked eye. The need for no adhesive has the further advantage of eliminating any possible contamination of the fragrance scent by any scent peculiar to the adhesive.

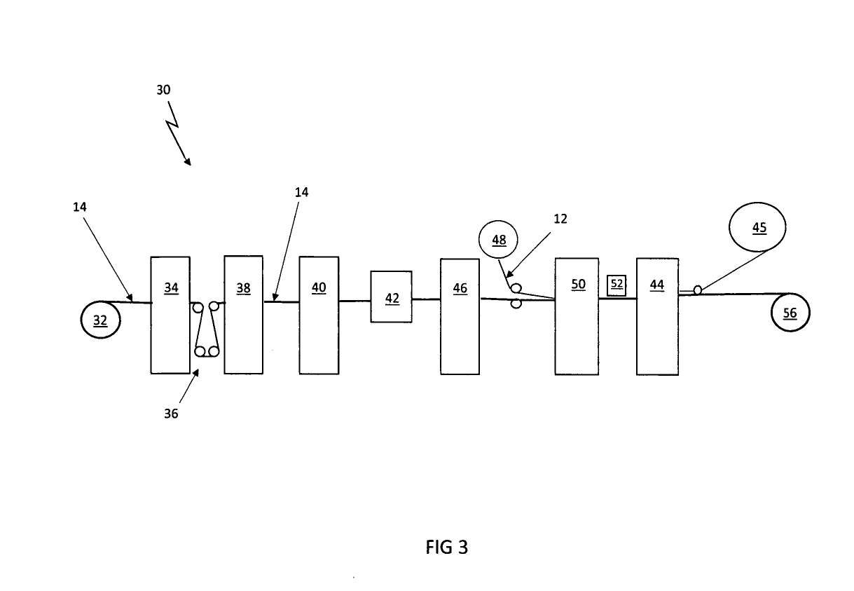

The method of manufacture is illustrated in FIG. 3, which is a schematic of a printing line 30 which prints text and graphics and coats the substrate with fragrance layers, dries the substrate, applies the upper substrate, then cures the entire construction before the web is rewound on a take up roll. The lower substrate 14 in the form of a one peelable label width, together with an adhesive layer 15 and liner 17, is withdrawn from a roll 32 and enters a print unit 34. As previously stated, the lower or bottom substrate 14 can be a polymer film, such as polypropylene, or it can be an alternative material, such as a heavy weight paper or the like. In the printing station 34, the necessary graphics and text desired is printed on the substrate. Additionally, if a cue mark or cue line is required on the web to serve as an indicator of separate labels and the location for later affixing those labels, it would be printed on the opposite side of the lower substrate in printing station 38. The web would undergo a series of turn bars 36 to reverse the web side for any printing required on the reverse side of the lower substrate 14, or to be accomplished in print station 38. Once the printing has been accomplished on the base or lower substrate, then a water based fragrance slurry is introduced into fragrance coating station 40 and applied by a roller to the lower substrate 14. The lower substrate 14 and first slurry coat then enters a hot air dryer 42 for drying the first slurry layer. The web then enters a second coating station 46 where a UV based fragrance slurry is applied over the dried water based slurry. The web exits the second fragrance coating station and the top ply substrate 12, such as a transparent polypropylene film, is then drawn from a separate roller unit 48 and conjoined with the bottom ply substrate 14 in a nip roller unit 50. The conjoined construction of upper ply substrate 12, lower ply substrate 14, and the coating layers is then introduced into an ultraviolet heat lamp area 52 which cures the UV based fragrance slurry, dries it, and causes the two slurry coats and top substrate 12 to meld or bond together. The UV curing process is essential for the “two part” fragrance design to perform as intended. When the upper substrate 12 is subsequently peeled from the label using the uncoated corner 20, the fragrance bead capsules of the two coated layers which have bonded together, will fracture, releasing the fragrance. However, not all of the fragrance beads will fracture, and those that do not can be scratched to cause their rupture for a subsequent release of the scent.

After UV curing, the web enters a die cut station 44 and a “kiss cut” die cuts the dual substrate down to the liner to form the peel back fragrance label with rounded corners as illustrated in FIG. 2, or could similarly die cut the web to form a peel back fragrance label of any shape desired by the ultimate user. The excess matrix of dual substrates and adhesive 17 and 15, which is separated in the die cut station 44, resembles a ladder structure having longitudinal side strips and intermittent cross strips. This matrix of excess material is taken up in a matrix collection roll 45 for disposal. The cured web containing the peelable fragrance labels on release liner is then wound onto a take up reel 56.

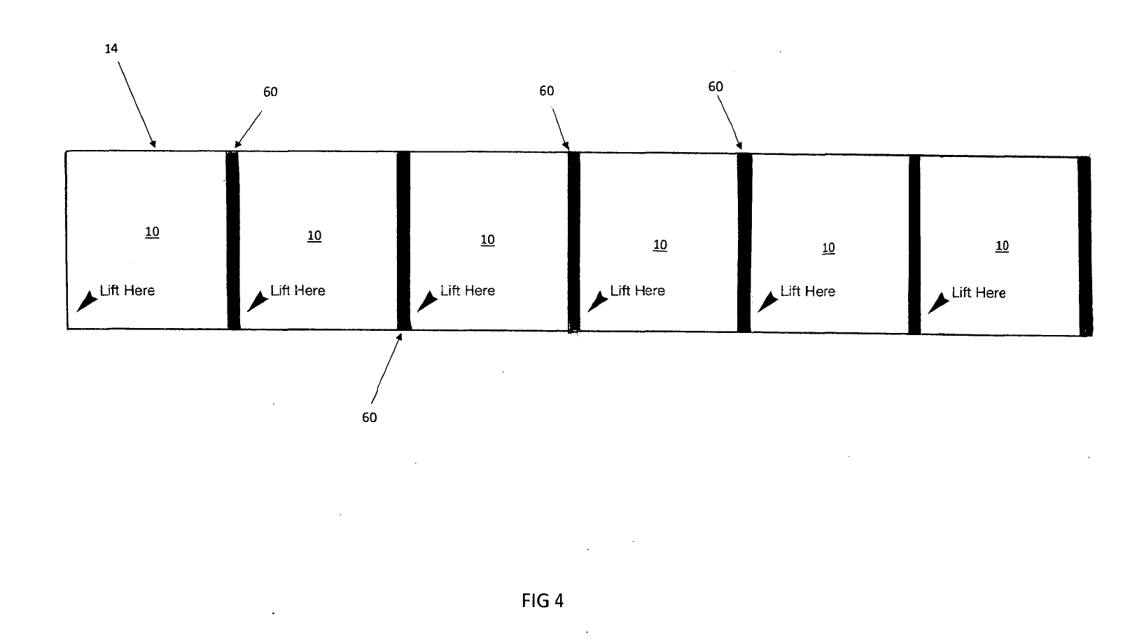

The process of manufacture in FIG. 3 was explained with respect to a narrow web flexographic printing press. FIG. 4 is an illustration of that web, a single label width, single lane output on the web/press. The web is one peelable label wide and is formed with a series of cue marks or cue lines 60 which delineate separate peelable labels. FIG. 4 also illustrates the manner in which graphics or text have been printed on the lower substrate 14, which include directions for its opening. The cue marks or cue lines 60 assist in the separation of the peelable labels from a release liner 17 for automated application in a later process. The cue marks could be a mark or line printed between each label, that would later be read by an electronic eye or scanner to activate the automated label affixing equipment. The activation may also be initiated by equipment that can read the difference in density of the labels, distinguishing the thinner “liner” area of less thickness between each label, than the thicker area of the 2-ply substrate label on top of the liner thickness, thereby determining the location of each individual label, and the subsequent processing equipment can either read the printed cue or detect the difference in thickness.

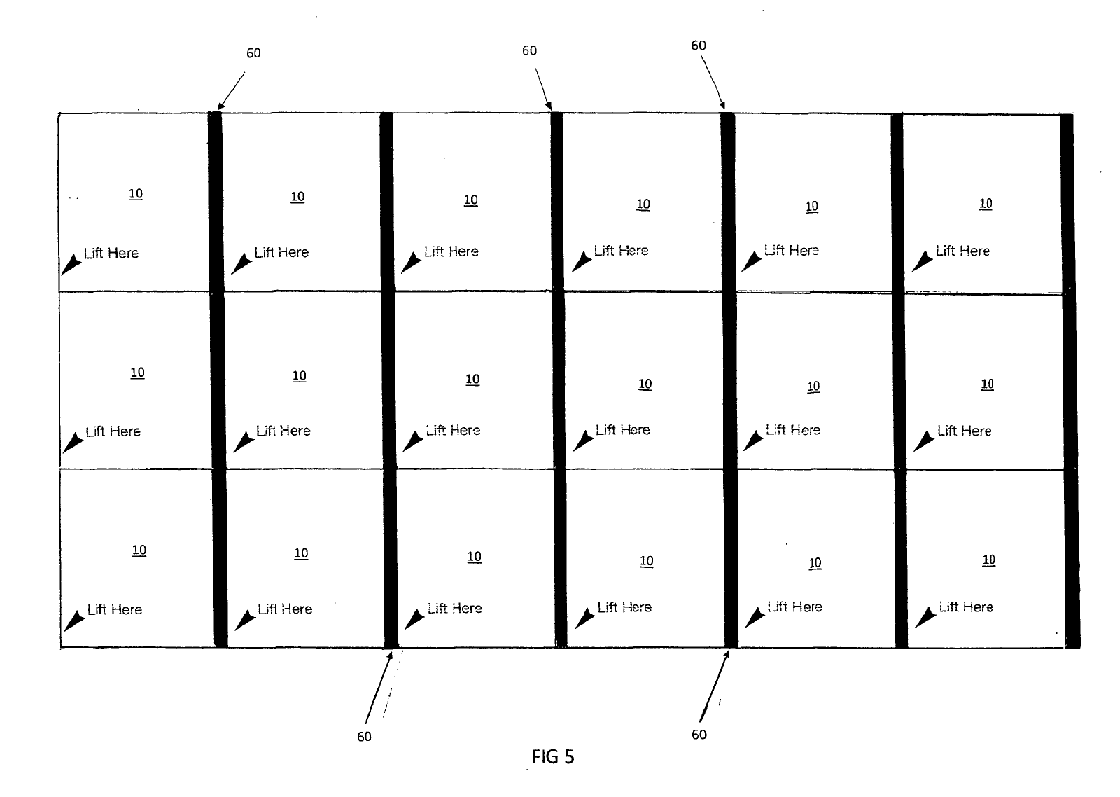

The peelable labels could also be fabricated in or on a web substrate to produce multiple labels/lanes across as illustrated in FIG. 5. The manufacturing process would be identical with the exception that the printing presses could be of a wider width to accommodate the greater width of rolls of substrate. Narrow web flexography presses can also produce multiple lanes across the width, depending on the size of the labels. In this particular format, the cue marks 60 would be printed in multiple lanes across the web substrate, as well as indicia copy 62. The subsequent slitting of the web substrate to separate the lanes of labels and rewind onto individual one across rolls, would need to take place off-press as a secondary manufacturing operation, which is a common practice for producing multiple lanes of labels across in master rolls.

### Formulation Example

The fragrance oil utilized in the peelable label may be selected from a variety of stock of fragrance oils, or made special. The fragrance oils may be used singularly, or in a mixture to create a specific character or fragrance. The fragrance oil should be formulated with as little volatile or water soluable reactive ingredients as possible to enhance and enable the encapsulation process, which is the forming of the cell wall around the fragrance oil creating beads or microencapsulated fragrance. The fragrance formulation or slurry would be composed of the microencapsulated fragrance oil and a transporter fluid for the purpose of transporting the encapsulated fragrance through the unit of application and onto the host substrate, the transporter fluid will later be dried and flashed-off when exposed to the UV heat lamp in the final drying/curing process. The microencapsulated fragrance coats will bond with the inner surfaces of the top and bottom substrates between which it is sandwiched and remain intact until the peel back fragrance label is opened. At that time a portion of the capsules will be broken open as the upper ply substrate 12 is peeled back, releasing the scent of the fragrance oil. Not all of the microencapsulated fragrance will rupture upon removal of the top ply of peel back fragrance label, and some of the microencapsulated fragrance beads will remain intact, thus causing a further release of the fragrance and scent if desired by scratching or rubbing the coated layers on the lower substrate to rupture these remaining beads and release the fragrance or oil for additional sampling usage.

The mixture of ingredients and their ratio can vary when creating the water based fragrance encapsulation layer 16, however, it may include a polyvinyl acetate, which is a type of thermoplastic or vinyl polymer, water, carboxymethyl cellulose or gum arabic, which would act as a binder. The mixing time can vary depending upon the fragrance oil composition, but may take approximately 24 to 26 hours for the cell walls to form and encapsulate the fragrance oil. The solution then undergoes a filtration process that may include straining through a fine mesh in order to drain off a majority of the liquid. The ratio of the fragrance capsules to (PVA) transporter can vary, but may be as much a 1:1 ratio with equal parts of the fragrance capsules and the PVA water based transporter. The ratio of fragrance capsules to UV transporter fluid in the UV fragrance layer 16 can vary, but may be as much as a 1:3 ratio of capsules to UV based transporter. In turn, the wet capsules can be further dried if required to create “Dry Capsules”. Dry capsules can eventually be process into a fine powder, by a number of processes including but not limited to sifting with a fine mesh screen. Dry fragrance capsules are mixed with a (UV) ultra violet transporter, to create fragrance coat 18 for application in the fragrance print unit 46. The ratio of dry fragrance capsules to (UV) transporter can vary, but may be such as a 1:3 ratio of plastic capsules to (UV) based transporter. In fragrance (print) unit 46 the (UV) based fragrance coat 18 is applied wet on top of the dried water based fragrance foundation area 16. The top polypropylene label ply 12 is then applied, and joined together with the bottom ply 14 in the nip roller unit 50. The two part fragrance within the construction is then cured by heating with a UV lamp 50 drying both fragrance parts 1 and 2 to each other, and to the inner sides of the top and bottom ply substrates.

The individual fragrance label illustrated in FIG. 2 is shown as a generally square label having die cut rounded edges. It will be recognized by those of skill in the art that the peelable fragrance label is not limited to this geometric shape or corner shape, but can be modified depending upon the product for which the fragrance or scent is being tested to further advertise the product. For example if a company whose name was a particular fruit wished to produce peelable fragrance labels for sampling with respect to a new flavored and scented juice, the labels could be manufactured in the form of the product or the particular fruit shape, or can having text or graphics which would further advertise the product.

Therefore, while the present invention has been disclosed with respect to the preferred embodiments thereof, it will be recognized by those of ordinary skill in the art that various changes and modifications can be made without departing from the spirit and scope of the invention. It is therefore manifestly intended that the invention be limited only by the claims and the equivalence thereof.

We claim:

1. A peel back fragrance label for the delivery of a scent or fragrance, the peel back fragrance label comprising:
a first ply substrate and a second ply substrate of equal size, said first ply substrate comprising the top substrate and said second ply substrate comprising of the bottom substrate, the bottom substrate having coated thereon a first fragrance coat comprising microscopic beads of fragrance, scent, or scented oil, encapsulated in a first transfer medium and a second fragrance coat comprising microscopic beads of fragrance, scent, or scented oil, encapsulated on a second transfer medium, said first and second fragrance coats upon drying sufficiently bond said first top ply to said second bottom ply encasing said fragrance coats.

2. The peel back fragrance label in accordance with claim 1 wherein said first top ply substrate positioned on said bottom ply substrate encasing said fragrance coats allows said fragrance coats to bond to said plies.

3. The peel back fragrance label in accordance with claim 1 wherein said first top ply substrate and said second bottom ply substrate are separable by means of a corner tab, said separation fracturing and releasing a portion of said fragrance, scent or scented microencapsulated oils from said fragrance coats.

4. The peel back fragrance label in accordance with claim 3 wherein a portion of said fragrance scent or microencapsulated oils not released by fracturing may be released subsequently by rubbing said coats.

5. The peel back fragrance label in accordance with claim 1 wherein said first ply substrate and said second ply substrate are formed from polypropylene.

6. The peel back fragrance label in accordance with claim 1 wherein said first ply substrate and said second ply substrate are formed from paper stock and a laminate such as but not limited to a polymer film.

7. The peel back fragrance label in accordance with claim 1 wherein an outer surface said second ply substrate is formed with an adhesive layer and overlying release liner allowing said peel back fragrance label to be attached to an article.

8. A peel back fragrance label for delivery of a scent or fragrance in accordance with claim 1 wherein said first fragrance coat comprising microscopic beads of fragrance, scent, or scented oil is encapsulated in a first transfer medium which is water based and heat dried.

9. A peel back fragrance label for delivery of a scent or fragrance in accordance with claim 1 wherein said second fragrance coat comprising microscopic beads of fragrance, scent, or scented oil is encapsulated in a second UV based transfer medium which is UV dried.

10. A method of fabricating a peel back fragrance label which employs an encapsulated scent or fragrance, for release upon opening of said peel back fragrance label, the method comprising the following steps:
(a) positioning a roll of second ply substrate in alignment with a printing press;
(b) unwinding said roll into a linear web and passing said linear web through a plurality of print rollers thereby forming an indicia and coloring on said linear web identifying a bottom ply of a peel back fragrance label;
(c) drying said indicia on said web;
(d) positioning a slurry of fragrance or scent in a reservoir in registration with said linear web;
(e) transferring a portion of said slurry of fragrance or scent onto said web in the form of a first fragrance coat in registration with said web;
(f) drying said first fragrance coat by hot air, bonding said first fragrance coat to said bottom ply;
(g) positioning a second slurry of fragrance or scent in registration with said web;
(h) transferring a portion of said slurry of fragrance or scent onto said web in the form of a second fragrance coat in registration with said web on top of said first fragrance coat;
(i) mating said web of said first ply substrate over said second fragrance coat;
(j) drying said second fragrance coat by UV light,
bonding said second fragrance coat to said first fragrance coat and simultaneously bonding said second ply bottom substrate to said first ply upper substrate; and
(k) die cutting said web to form successive peel back fragrance labels defined by the indicia and coloring of step (b) and having said defined fragrance coats in accordance with steps (d through j).

11. The method in accordance with claim 8 wherein adhesive layer and release liner may be preformed in registration to said upper surface of said lower second ply for attaching said peel back fragrance label to an article.

12. The method in accordance with claim 8 wherein said roll is formed of a narrow web and subjected to flexographic printing presses allowing the side by side fabricating of a top ply and bottom ply forming said peel back fragrance label, said application of said fragrance coats and said adhesive being to said bottom ply, said top ply being secured to said bottom ply during said UV drying process.
